(19) United States
(12) Patent Application Publication
(10) Pub. No.: US 2022/0380449 A1
(43) Pub. Date: Dec. 1, 2022

(54) METHODS OF TREATING NONALCOHOLIC FATTY LIVER DISEASE (NAFLD) USING IL-17RA ANTIBODY

(71) Applicant: BAUSCH HEALTH IRELAND LIMITED, Dublin (IE)

(72) Inventor: Robert J. Israel, Suffern, NY (US)

(21) Appl. No.: 17/642,130

(22) PCT Filed: Sep. 11, 2020
(86) PCT No.: PCT/EP2020/075477

§ 371 Date: Mar. 10, 2022

Related U.S. Application Data
(60) Provisional application No. 62/898,903, filed on Sep. 11, 2019.

(51) Int. Cl. C07K 16/24; A61P 1/16

(52) U.S. Cl. CPC C07K 16/244; A61P 1/16; A61K 2039/505

(57) ABSTRACT

The present disclosure is directed to a method of treating nonalcoholic fatty liver disease (NAFLD), and subsets thereof such as nonalcoholic steatohepatitis (NASH), using an IL-17 antagonist, such as a monoclonal antibody that specifically binds to IL-17 receptor A (IL-17RA).

### BACKGROUND

Nonalcoholic fatty liver disease (NAFLD) is a rapidly growing epidemic worldwide and is an increasingly important etiology of chronic liver disease and hepatocellular carcinoma (HCC) (Younossi et al., Hepatology, 62:1723-1730 (2015)); Younossi et al., Hepatology, 64(1):73-84 (2016); Wong, et al., Hepatology, 59(6): 2188-2195 (2014); Goldberg et al., Gastroenterology, 152(5): 1090-1099 (2017)). NAFLD encompasses a spectrum of liver abnormalities, ranging from simple steatosis, which is relatively benign, to nonalcoholic steatohepatitis (NASH), which is predicted to become the leading indication of liver transplantation by 2020 (Charlton et al., Clin Gastro & Hep., 2(12): 1048-1058 (2004)). NAFLD affects approximately 90 million people in the U.S. (Younossi et al., Gastroenterology, 150(8): 1778-1785 (2016)).

A multiple-hit hypothesis has been proposed for the development of NAFLD in the genetically susceptible individual. Dietary and environmental factors, together with obesity, result in metabolic perturbations that lead to raised serum levels of free fatty acids and cholesterol, development of insulin resistance, adipocyte proliferation, and changes in the intestinal microbiome. Hepatic steatosis resulting in lipotoxicity triggers the activation of inflammatory cascades, leading to hepatic inflammation, fibrosis, cirrhosis, and HCC.

Interleukin (IL)-17A (a ligand) belongs to a family of proinflammatory cytokines, produced in several organs including skin, mucosal tissues, and the liver. IL-17RA (the IL-17A receptor) is ubiquitously expressed by hepatocytes, Kupfer cells, hepatic stellate cells, biliary epithelial cells, and sinusoidal endothelial cells. IL-17-driven activation of IL-17RA results in the production of pro-inflammatory cytokines and neutrophil recruiting chemokines. Increased IL-17A production has been reported in various chronic liver diseases, including chronic hepatitis B and C, HCC, and alcoholic liver injury. Increased IL-17 expression has also been reported in obese humans and mice, and has been implicated in regulating obesity and NAFLD.

In animal models, IL-17A is the main family member of IL-17 in driving the pathogenesis of NAFLD (Harley et al., Hepatology, 59(5): 1830-1839 (2014); Xu et al., Acta Biochem Biophys., 45(9): 726-733 (2013)). In addition, a murine model of NASH exhibits significantly increased IL-17A expression compared to wild-type mice, as well as increased differentiation of macrophages to a pro-inflammatory phenotype that has been associated with hepatic inflammation and hepatocellular injury (Giles et al., PLOS One, 11(2): e0149783 (2016)). Thus, the IL-17 pathway has been implicated in the pathogenesis of NAFLD in animal models. At present, there are no Food and Drug Administration (FDA) approved medical treatments for NAFLD.

There remains a need for compositions and methods for treating NAFLD and subsets thereof, such as NASH.

### BRIEF SUMMARY OF THE INVENTION

The disclosure provides a method of treating nonalcoholic fatty liver disease (NAFLD) in a subject, which comprises administering to the subject a composition comprising a therapeutically effective amount of an IL-17 antagonist, such as a monoclonal antibody that specifically binds to interleukin 17 receptor A (IL-17RA), or an antigen-binding fragment thereof, and a pharmaceutically acceptable carrier, whereby the NAFLD is treated in the subject.

The disclosure also provides a method of reducing liver inflammation in a subject in need thereof, which comprises administering to the subject a composition comprising a therapeutically effective amount of an IL-17 antagonist, such as a monoclonal antibody that specifically binds to interleukin 17 receptor A (IL-17RA), or an antigen-binding fragment thereof, and a pharmaceutically acceptable carrier, whereby liver inflammation in the subject is reduced.

### BRIEF DESCRIPTION OF THE DRAWING(S)

FIG. 1 is a schematic diagram illustrating the clinical protocol described in Example 2.

### DETAILED DESCRIPTION

The present disclosure is predicated, at least in part, on the discovery that liver inflammation, particularly that associated with nonalcoholic fatty liver disease (NAFLD), may be treated via antagonism of IL-17 receptor A (IL-17RA).

### Definitions

To facilitate an understanding of the present technology, a number of terms and phrases are defined below. Additional definitions are set forth throughout the detailed description.

The term “immunoglobulin” or “antibody,” as used herein, refers to a protein that is found in blood or other bodily fluids of vertebrates, which is used by the immune system to identify and neutralize foreign objects, such as bacteria and viruses. Typically, an immunoglobulin or antibody is a protein that comprises at least one complementarity determining region (CDR). The CDRs form the “hypervariable region” of an antibody, which is responsible for antigen binding (discussed further below). A whole immunoglobulin typically consists of four polypeptides: two identical copies of a heavy (H) chain polypeptide and two identical copies of a light (L) chain polypeptide. Each of the heavy chains contains one N-terminal variable (V_{H}) region and three C-terminal constant (C_{H}1, C_{H}2, and C_{H}3) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. In a typical antibody, each light chain is linked to a heavy chain by disulphide bonds, and the two heavy chains are linked to each other by disulphide bonds. The light chain variable region is aligned with the variable region of the heavy chain, and the light chain constant region is aligned with the first constant region of the heavy chain. The remaining constant regions of the heavy chains are aligned with each other.

The variable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region which are located between the CDRs. There are four framework regions in each variable domain, which are designated FR1, FR2, FR3, and FR4. The framework regions form the β sheets that provide the structural framework of the variable region (see, e.g., C. A. Janeway et al. (eds.), Immunobiology, 5th Ed., Garland Publishing, New York, N.Y. (2001)).

The framework regions are connected by three CDRs. As discussed above, the three CDRs, known as CDR1, CDR2, and CDR3, form the “hypervariable region” of an antibody, which is responsible for antigen binding. The CDRs form loops connecting, and in some cases comprising part of, the beta-sheet structure formed by the framework regions. While the constant regions of the light and heavy chains are not directly involved in binding of the antibody to an antigen, the constant regions can influence the orientation of the variable regions. The constant regions also exhibit various effector functions, such as participation in antibody-dependent complement-mediated lysis or antibody-dependent cellular toxicity via interactions with effector molecules and cells.

As used herein, when an antibody or other entity (e.g., antigen binding domain) “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Ka) of at least 10⁷ M⁻¹ (e.g., >10⁷ M⁻¹, >10⁸ M⁻¹, >10⁹ M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

The terms “fragment of an antibody,” “antibody fragment,” and “antigen-binding fragment” of an antibody are used interchangeably herein to refer to one or more fragments of an antibody that retain the ability to specifically bind to an antigen (see, generally, Holliger et al., Nat. Biotech., 23(9): 1126-1129 (2005)). Any antigen-binding fragment of the antibody described herein is within the scope of the invention. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the V_{L}, V_{H}, C_{L}, and C_{H}1 domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a domain antibody (dAb), which is an antibody single variable region domain (V_{H} or V_{L}) polypeptide that specifically binds antigen.

The terms “nucleic acid,” “polynucleotide,” “nucleotide sequence,” and “oligonucleotide” are used interchangeably herein and refer to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The terms encompass any deoxyribonucleotide, ribonucleotide, or peptide nucleic acid component, and any chemical variants thereof, such as methylated, hydroxymethylated, or glycosylated forms of these bases. The polymers or oligomers may be heterogenous or homogenous in composition, may be isolated from naturally occurring sources, or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. The terms “nucleic acid” and “nucleic acid sequence” may also encompass a chain comprising non-natural nucleotides, modified nucleotides, and/or non-nucleotide building blocks that can exhibit the same function as natural nucleotides (e.g., “nucleotide analogs”).

The terms “peptide,” “polypeptide,” and “protein” are used interchangeably herein and refer to a polymeric form of amino acids of any length, which can include coded and non-coded amino acids, chemically or biochemically modified or derivatized amino acids, and polypeptides having modified peptide backbones.

The terms “immunogen” and “antigen” are used interchangeably herein and refer to any molecule, compound, or substance that induces an immune response in an animal (e.g., a mammal). An “immune response” can entail, for example, antibody production and/or the activation of immune effector cells. An antigen in the context of the disclosure can comprise any subunit, fragment, or epitope of any proteinaceous or non-proteinaceous (e.g., carbohydrate or lipid) molecule that provokes an immune response in a mammal. By “epitope” is meant a sequence of an antigen that is recognized by an antibody or an antigen receptor. Epitopes also are referred to in the art as “antigenic determinants.” In certain embodiments, an epitope is a region of an antigen that is specifically bound by an antibody. In certain embodiments, an epitope may include chemically active surface groupings of molecules such as amino acids, sugar side chains, phosphoryl, or sulfonyl groups. In certain embodiments, an epitope may have specific three-dimensional structural characteristics (e.g., a “conformational” epitope) and/or specific charge characteristics. An antigen can be a protein or peptide of viral, bacterial, parasitic, fungal, protozoan, prion, cellular, or extracellular origin, which provokes an immune response in a mammal, preferably leading to protective immunity.

### IL-17 Antagonists

The methods described herein utilize an IL-17 antagonist, e.g., an IL-17-binding molecule (e.g., a soluble IL-17 receptor or an IL-17-binding antibody or antigen-binding fragment thereof) or an IL-17 receptor-binding molecule (e.g., an IL-17 receptor-binding antibody or antigen-binding fragment thereof). Examples of 17-binding antibodies that may be used in the described methods include, but are not limited to, secukinumab (COSENTYX®), ixekizumab (TALTZ®), and CJM112 (see, e.g., Riis et al., Expert Opin. Investig. Drugs, 27(1): 43-53 (2018)). IL-17 antagonists are further described in e.g., Wasilewska et al., Postepy. Dermatol., Alergol., 33(4): 247-252 (2016); and Silfvast-Kaiser et al., Expert Opin. Biol. Ther., 19(1): 45-54 (2019). In some embodiments, the IL-17 antagonist is an antibody or antigen-binding fragment thereof which specifically binds to IL-17 receptor A (“IL-17RA”). The terms “IL-17 receptor A,” “IL-17RA,” “IL-17 receptor,” and “IL-17R” are used interchangeably herein to refer to the cell surface receptor and receptor complexes (e.g., the IL-17RA-IL-17RC complex and IL-17RA-IL-17RB) that bind to the cytokine IL-17A. IL-17A is an inflammatory cytokine initially identified as a transcript selectively expressed by activated T cells. IL-17RA is a ubiquitously expressed and shown to bind IL-17A with an affinity of approximately 0.5 nM (Yao et al., Immunity, 3: 811-821 (1995)). Five additional IL-17-like ligands (i.e., IL-17B, IL-17C, IL-17D, IL-17E, and IL-17F) and four additional IL-17RA-like receptors (i.e., IL-17RB, IL-17RC, IL-17RD, and IL-17RE) have been identified (Kolls and Linden, Immunity, 21: 467-476 (2004)). Different IL-17RA receptor complexes are known to bind one or more of the various IL-17 ligands, thereby initiating a signal transduction pathway within the cell. The cloning, characterization, and preparation of IL-17RA is described in, for example, U.S. Pat. No. 6,072,033.

The antibody or antigen-binding fragment thereof described herein may specifically bind to full length, wild type IL-17RA, the amino acid sequence of which is shown in SEQ ID NO: 9. Alternatively, the antibody or antigen-binding fragment thereof may specifically bind to variants, mutants, and/or fragments of IL-17RA. Such variants, mutants, and/or fragments of IL-17RA desirably retain the ability to bind to IL-17A and/or IL-17F. In some embodiments, for example, the antibody or antigen-binding fragment thereof may specifically bind to the extracellular domain of IL-17RA or a mature form of IL-17RA which lacks the signal peptide. The antibody or antigen-binding fragment thereof may specifically bind to any IL-17RA mutant or variant having an amino acid sequence that is between about 70% and 99% identical to SEQ ID NO: 9 and as described in U.S. Pat. No. 6,072,033, so long as the IL-17RA mutant or variant retains the capacity to bind IL-17A and/or IL-17F, or a heteromeric version of IL-17A and/or IL-17F. In other embodiments, the antibody or antigen-binding fragment thereof may specifically bind to an IL-17RA protein comprising post-translational modifications, such as, for example, N-and O-linked glycosylation.

The antibody or an antigen-binding fragment thereof disclosed herein comprises a heavy chain variable region comprising a complementarity determining region 1 (CDR) amino acid sequence of SEQ ID NO: 1, a CDR2 amino acid sequence of SEQ ID NO: 2, and a CDR3 amino acid sequence of SEQ ID NO: 3, and a light chain variable region comprising a CDR1 amino acid sequence of SEQ ID NO: 4, a CDR2 amino acid sequence of SEQ ID NO: 5, and a CDR3 amino acid sequence of SEQ ID NO: 6. In other embodiments, the antibody or antigen-binding fragment thereof may comprise heavy chain variable region CDR1, CDR2, and CDR3 amino acid sequences that are at least 90% identical to SEQ ID NO: 1, SEQ ID NO: 2, and/or SEQ ID NO: 3, respectively, and light chain variable region CDR1, CDR2, and CDR3 amino acid sequences that are at least 90% identical to SEQ ID 4, SEQ ID NO: 5, and/or SEQ ID NO: 6, respectively.

In one embodiment, the heavy chain variable region (V_{H}) CDR1 amino acid sequence comprises, consists essentially of, or consists of SEQ ID NO: 1, the V_{H} CDR2 amino acid sequence comprises, consists essentially of, or consists of SEQ ID NO: 2, and the V_{H} CDR3 amino acid sequence comprises, consists essentially of, or consists of SEQ ID NO: 3. When the V_{H} CDR1, V_{H} CDR2, and V_{H} CDR3 amino acid sequences of the disclosed antibody consist essentially of SEQ ID NO: 1, SEQ ID NO: 2 and SEQ ID NO: 3, respectively, additional components can be included in the CDR that do not materially affect the antibody or antigen-binding fragment thereof (e.g., protein moieties such as biotin that facilitate purification or isolation). When the V_{H} CDR1, V_{H} CDR2, and V_{H} CDR3 amino acid sequences of the disclosed antibody consist of SEQ ID NO: 1, SEQ ID NO: 2, and SEQ ID NO: 3, respectively, each CDR does not comprise any additional components (i.e., components that are not endogenous to the CDR). Similarly, the light chain variable region (V_{L}) CDR1 amino acid sequence comprises, consists essentially of, or consists of SEQ ID NO: 4, the V_{L} CDR2 amino acid sequence comprises, consists essentially of, or consists of SEQ ID NO: 5, and the V_{L} CDR3 amino acid sequence comprises, consists essentially of, or consists of SEQ ID NO: 6. When the V_{L} CDR1, V_{L} CDR2, and V_{L} CDR3 amino acid sequences of the disclosed antibody consist essentially of SEQ ID NO: 4, SEQ ID NO: 5, and SEQ ID NO: 6, respectively, additional components can be included in the CDR that do not materially affect the antibody or antigen-binding fragment thereof (e.g., protein moieties such as biotin that facilitate purification or isolation). When the V_{L} CDR1, V_{L} CDR2, and V_{L} CDR3 amino acid sequences of the disclosed antibody consist of SEQ ID NO: 4, SEQ ID NO: 5, and SEQ ID NO: 6, respectively, each CDR does not comprise any additional components (i.e., components that are not endogenous to the CDR).

In some embodiments, the antibody or antigen-binding fragment thereof comprises a heavy chain variable region (V_{H}) amino acid sequence comprising, consisting essentially of, or consisting of SEQ ID NO: 7 and a light chain variable region (V_{L}) amino acid sequence comprising, consisting essentially of, or consisting of SEQ ID NO: 8. When the V_{H} amino acid sequence consists essentially of SEQ ID NO: 7 and the V_{L} amino acid sequence consists essentially of SEQ ID NO: 8, additional components can be included in the heavy or light chain variable regions that do not materially affect the antibody or antigen-binding fragment thereof (e.g., protein moieties such as biotin that facilitate purification or isolation). When the V_{H} amino acid sequence consists of SEQ ID NO: 7 and the V_{L} amino acid sequence consists of SEQ ID NO: 8, the heavy and light chain variable regions do not comprise any additional components (i.e., components that are not endogenous to the heavy or light chain variable region).

A human monoclonal antibody comprising a V_{H} amino acid sequence comprising SEQ ID NO: 7 and a V_{L} amino acid sequence comprising SEQ ID NO: 8 is marketed in the U.S. as SILIQ™ (brodalumab) by Ortho Dermatologics, Inc., and in Europe as KYNTHEUM® by LEO Pharma, Inc. Brodalumab is a human monoclonal antibody and IL-17 receptor antagonist recently approved by the FDA for the treatment of moderate to severe psoriasis. Brodalumab has been implicated in dampening inflammation associated with psoriasis and other chronic inflammatory disorders (Sherlock et al., Nat. Med., 18(7): 1069-1076 (2012)).

Brodalumab was approved for the treatment of psoriasis in 2017 based on Phase 3 clinical studies (Strober et al., Journal of the American Academy of Dennatology, 72(5): AB224 (2015); Lebwohl et al., N. Eng. J. Med., 373(14): 1318-1328 (2015)). Brodalumab is well tolerated and has a favorable safety profile. The most common treatment-emergent adverse events (TEAEs) observed in Phase 3 studies with brodalumab were nasopharyngitis, upper respiratory tract infections, headache, and arthralgia. Neutropenia was also observed but did not translate to serious infections. Furthermore, the neutropenia was mild, transient and reversible. There were more Candida infections observed in the brodalumab treatment group compared to placebo. Though suicidal ideation has been included as a boxed warning, further analysis of patients in the brodalumab program did not demonstrate evidence of causality (Lebwohl et al., Journal of the American Academy of Dermatology, 78(1): 81-89.e5 (2018)).

The disclosure also provides an antibody or antigen-binding fragment thereof which comprises a heavy chain variable region amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 7 and a light chain variable region amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 8. Nucleic acid or amino acid sequence “identity,” as described herein, can be determined by comparing a nucleic acid or amino acid sequence of interest to a reference nucleic acid or amino acid sequence. The percent identity is the number of nucleotides or amino acid residues that are the same (i.e., that are identical) as between the sequence of interest and the reference sequence divided by the length of the longest sequence (i.e., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

One or more amino acids of the aforementioned antibody or antigen-binding fragment thereof can be replaced or substituted with a different amino acid, so long as the antibody or antigen-binding fragment thereof retains the ability to specifically bind to IL-17RA. An amino acid “replacement” or “substitution” refers to the replacement of one amino acid at a given position or residue by another amino acid at the same position or residue within a polypeptide sequence.

Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gln), lysine (K or Lys), and arginine (R or Arg).

Aliphatic amino acids may be sub-divided into four sub-groups. The “large aliphatic non-polar sub-group” consists of valine, leucine, and isoleucine. The “aliphatic slightly-polar sub-group” consists of methionine, serine, threonine, and cysteine. The “aliphatic polar/charged sub-group” consists of glutamic acid, aspartic acid, asparagine, glutamine, lysine, and arginine. The “small-residue sub-group” consists of glycine and alanine. The group of charged/polar amino acids may be sub-divided into three sub-groups: the “positively-charged sub-group” consisting of lysine and arginine, the “negatively-charged sub-group” consisting of glutamic acid and aspartic acid, and the “polar sub-group” consisting of asparagine and glutamine.

Aromatic amino acids may be sub-divided into two sub-groups: the “nitrogen ring sub-group” consisting of histidine and tryptophan and the “phenyl sub-group” consisting of phenylalanine and tyrosine.

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra).

Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained.

“Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In addition, one or more amino acids can be inserted into the antibody or antigen-binding fragment thereof (e.g., insertion into the heavy and/or light chain variable region amino acid sequence), so long as the antibody or antigen-binding fragment thereof retains the ability to specifically bind to IL-17RA. Any number of any suitable amino acids can be inserted into the amino acid sequence of the antibody or antigen-binding fragment thereof. In this respect, at least one amino acid (e.g., 2 or more, 5 or more, or 10 or more amino acids), but not more than 20 amino acids (e.g., 18 or less, 15 or less, or 12 or less amino acids), can be inserted into the amino acid sequence of the antibody or antigen-binding fragment thereof. For example, 1-10 amino acids (e.g., 1, 2, 3, 4, 5, 6, 7, 8, 9, or 10 amino acids) may be inserted into the amino acid sequence of the antibody or antigen-binding fragment thereof. In this respect, the amino acid(s) can be inserted into antibody or antigen-binding fragment thereof in any suitable location. Preferably, the amino acid(s) are inserted into a CDR (e.g., CDR1, CDR2, or CDR3) of the antibody or antigen-binding fragment thereof.

The inventive antibody or antigen-binding fragment thereof is not limited to a polypeptide comprising the specific amino acid sequences described herein. Indeed, the antibody or antigen-binding fragment thereof can comprise any heavy chain polypeptide or light chain polypeptide that competes with the inventive antibody or antigen-binding fragment thereof for binding to IL-17RA. Antibody competition can be assayed using routine peptide competition assays such as, for example, ELISA, Western blot, or immunohistochemistry methods (see, e.g., U.S. Pat. Nos. 4,828,981 and 8,568,992; and Braitbard et al., Proteome Sci., 4: 12 (2006)).

The antibody or antigen-binding fragment thereof described herein desirably is monoclonal antibody. The term “monoclonal antibody,” as used herein, refers to an antibody produced by a single clone of B lymphocytes that is directed against a single epitope on an antigen. Monoclonal antibodies typically are produced using hybridoma technology, as first described in Köhler and Milstein, Eur. J. Immunol., 5: 511-519 (1976). Monoclonal antibodies may also be produced using recombinant DNA methods (see, e.g., U.S. Pat. No. 4,816,567), isolated from phage display antibody libraries (see, e.g., Clackson et al. Nature, 352: 624-628 (1991)); and Marks et al., J. Mol. Biol., 222: 581-597 (1991)), or produced from transgenic mice carrying a fully human immunoglobulin system (see, e.g., Lonberg, Nat. Biotechnol., 23(9): 1117-25 (2005), and Lonberg, Handb. Exp. Pharmacol., 181: 69-97 (2008)). In contrast, “polyclonal” antibodies are antibodies that are secreted by different B cell lineages within an animal. Polyclonal antibodies are a collection of immunoglobulin molecules that recognize multiple epitopes on the same antigen.

The IL-17RA-binding antibody or antigen-binding fragment thereof can be a human antibody, a non-human antibody, a chimeric antibody, or a humanized antibody. By “chimeric” is meant an antibody or fragment thereof comprising both human and non-human regions. A “humanized” antibody is a monoclonal antibody comprising a human antibody scaffold and at least one CDR obtained or derived from a non-human antibody. Non-human antibodies include antibodies isolated from any non-human animal, such as, for example, a rodent (e.g., a mouse or rat). A humanized antibody can comprise, one, two, or three CDRs obtained or derived from a non-human antibody. In certain embodiments, the IL-17RA-binding antibody or antigen-binding fragment thereof is a human monoclonal antibody.

A human antibody, a non-human antibody, a chimeric antibody, or a humanized antibody can be obtained by any means, including via in vitro sources (e.g., a hybridoma or a cell line producing an antibody recombinantly) and in vivo sources (e.g., rodents). Methods for generating antibodies are known in the art and are described in, for example, Köhler and Milstein, Eur. J. Immunol., 5: 511-519 (1976); Harlow and Lane (eds.), Antibodies: A Laboratory Manual, CSH Press (1988); and Janeway et al. (eds.), Immunobiology, 5th Ed., Garland Publishing, New York, N.Y. (2001)).

### Compositions and Formulations

The disclosure also provides a composition comprising an IL-17 antagonist, such as an IL-17RA-binding monoclonal antibody or antigen-binding fragment thereof described herein. The composition desirably is a pharmaceutically acceptable (e.g., physiologically acceptable) composition, which comprises a carrier, preferably a pharmaceutically acceptable (e.g., physiologically acceptable) carrier, and the monoclonal antibody or antigen-binding fragment thereof. Any suitable carrier can be used within the context of the disclosure, and such carriers are well known in the art. For example, the composition may contain preservatives, such as, for example, methylparaben, propylparaben, sodium benzoate, and benzalkonium chloride. A mixture of two or more preservatives optionally may be used. In addition, buffering agents may be included in the composition. Suitable buffering agents include, for example, glutamic acid (glutamate), citric acid, sodium citrate, phosphoric acid, potassium phosphate, and various other acids and salts. A mixture of two or more buffering agents optionally may be used. Methods for preparing compositions for pharmaceutical use are known to those skilled in the art and are described in, for example, Remington: The Science and Practice of Pharmacy, Lippincott Williams & Wilkins; 21st ed. (May 1, 2005).

The composition desirably comprises a “therapeutically effective amount” of the IL-17 antagonist, such as an IL-17RA-binding monoclonal antibody or antigen-binding fragment thereof. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the antibody to elicit a desired response in the individual. For example, a therapeutically effective amount of an IL-17RA-binding monoclonal antibody of the invention is an amount which decreases IL-17A and/or IL-17F bioactivity in a human and/or reduces liver inflammation.

Alternatively, the pharmacologic and/or physiologic effect may be prophylactic, i.e., the effect completely or partially prevents a disease or symptom thereof. In this respect, the inventive method comprises administering a “prophylactically effective amount” of the IL-17 antagonist (e.g., an IL-17RA-binding antibody). A “prophylactically effective amount” refers to an amount effective, at dosages and for periods of time necessary, to achieve a desired prophylactic result (e.g., prevention of inflammation, psoriasis, nonalcoholic fatty liver disease (NAFLD), or nonalcoholic steatohepatitis (NASH)).

A typical dose of antibody can be, for example, in the range of 0.1 μg/kg to 30 mg/kg of animal or human body weight; however, doses below or above this exemplary range are within the scope of the invention. For example, a daily parenteral dose can be about 0.2 μg/kg to about 25 mg/kg of total body weight (e.g., about 0.5 μg/kg, about 1.5 μg/kg, about 5 μg/kg, about 10 μg/kg, about 100 μg/kg, about 500 μg/kg, about 1 mg/kg, about 5 mg/kg, about 10 mg/kg, about 20 mg/kg, or a range defined by any two of the foregoing values), preferably from about 0.1 μg/kg to about 10 mg/kg of total body weight (e.g., about 0.5 μg/kg, about 1 μg/kg, about 50 μg/kg, about 150 μg/kg, about 300 μg/kg, about 750 μg/kg, about 1.5 mg/kg, about 5 mg/kg, or a range defined by any two of the foregoing values), more preferably from about 1 μg/kg to 5 mg/kg of total body weight (e.g., about 3 μg/kg, about 15 μg/kg, about 75 μg/kg, about 300 μg/kg, about 900 μg/kg, about 1 mg/kg, about 2 mg/kg, about 4 mg/kg, or a range defined by any two of the foregoing values). In specific embodiments, the dosage may range from 0.1 μg/kg up to about 30 mg/kg, optionally from 1 μg/kg to about 30 mg/kg or from 10 μg/kg to about 5 mg/kg. In certain embodiments, the method comprises administering a total daily dose of about 150 mg to about 250 mg (e.g., about 160 mg, about 170 mg, about 180 mg, about 190 mg, about 200 mg, about 210 mg, about 220 mg, about 230 mg, or about 240 mg) of the monoclonal antibody.

Formulations containing IL-17RA-binding monoclonal antibodies, such as brodalumab, are described in, e.g., U.S. Pat. No. 10,072,085, and such formulations are within the scope of the present disclosure. In one embodiment, the disclosure provides a formulation comprising about 100 to 150 mg/mL of the IL-17RA-binding monoclonal antibody (e.g., about 110 mg/mL, about 120 mg/mL, about 130 mg/mL, or about 140 mg/mL of antibody), about 5 mM to about 30 mM glutamate (e.g., about 10 mM, about 15 mM, about 20 mM, or about 25 mM glutamate), 2-4% proline (e.g., about 2.5%, about 3.0%, or about 3.5% proline), and 0.001-0.02% (w/v) polysorbate 20 (e.g., about 0.005%, about 0.05%, or about 0.015% proline) at pH of about 4.4 to about 5.2 (e.g., about 4.5, about 4.6, about 4.7, about 4.8, about 4.9, about 5.0, or about 5.1). For example, the formulation may comprise 1.5 mL (210 mg) of brodalumab, formulated with 10 mM L-glutamate, 3% (w/v) L-proline, and 0.001% (w/v) polysorbate 20, at pH 4.8

Therapeutic or prophylactic efficacy can be monitored by periodic assessment of treated patients. For repeated administrations over several days or longer, depending on the condition, the treatment can be repeated until a desired suppression of disease symptoms occurs. However, other dosage regimens may be useful and are within the scope of the invention. The desired dosage can be delivered by a single bolus administration of the composition, by multiple bolus administrations of the composition, or by continuous infusion administration of the composition, as discussed further below.

The disclosure further provides a nucleic acid sequence encoding the aforementioned antibody or antigen-binding fragment thereof. In certain embodiments, the nucleic acid sequence is in the form of a vector. The vector can be, for example, a plasmid, episome, cosmid, viral vector (e.g., retroviral or adenoviral), or phage. Suitable vectors and methods of vector preparation are well known in the art (see, e.g., Sambrook et al., Molecular Cloning, a Laboratory Manual, 4th edition, Cold Spring Harbor Press, Cold Spring Harbor, N.Y. (2012), and Ausubel et al., Current Protocols in Molecular Biology, Greene Publishing Associates and John Wiley & Sons, New York, N.Y. (1994)).

In addition to the nucleic acid encoding the antibody or antigen-binding fragment thereof, the vector desirably comprises expression control sequences, such as promoters, enhancers, polyadenylation signals, transcription terminators, internal ribosome entry sites (IRES), and the like, that provide for the expression of the antibody-encoding nucleic sequence in a host cell. Exemplary expression control sequences are known in the art and described in, for example, Goeddel, Gene Expression Technology: Methods in Enzymology, Vol. 185, Academic Press, San Diego, Calif. (1990).

The monoclonal antibody or antigen-binding fragment thereof can be provided in a kit, i.e., a packaged combination of reagents in predetermined amounts with instructions for performing a method of using the antibody (e.g., a method of treating NAFLD in a subject). As such, the disclosure provides a kit comprising the monoclonal antibody or antigen-binding fragment described herein and instructions for use thereof (e.g., use for treating liver inflammation or NAFLD). The instructions can be in paper form or computer-readable form, such as a disk, CD, DVD, etc. Alternatively or additionally, the kit can comprise a calibrator or control, and/or at least one container, and/or a buffer. Ideally, the kit comprises all components, i.e., reagents, standards, buffers, diluents, etc., which are necessary to perform the method. Other additives may be included in the kit, such as stabilizers, buffers (e.g., a blocking buffer or lysis buffer), and the like. The relative amounts of the various reagents can be varied to provide for concentrations in solution of the reagents which substantially optimize the method. The reagents may be provided as dry powders (typically lyophilized), including excipients which on dissolution will provide a reagent solution having the appropriate concentration.

### Treatment Methods

The disclosure provides a method of treating nonalcoholic fatty liver disease (NAFLD), nonalcoholic steatohepatitis (NASH), and/or liver inflammation in a subject, which comprises administering an effective amount of the above-described composition comprising a therapeutically effective amount of an IL-17 antagonist, such as an IL-17RA-binding monoclonal antibody, and a pharmaceutically acceptable carrier to a subject in need thereof. As used herein, the terms “treatment,” “treating,” and the like refer to obtaining a desired pharmacologic and/or physiologic effect. Preferably, the effect is therapeutic, i.e., the effect partially or completely cures a disease and/or adverse symptom attributable to the disease. As discussed above, the term “nonalcoholic fatty liver disease” (NAFLD), as used herein, is a generic term that refers to a range of liver conditions characterized by the storage of excessive fat in liver cells not caused by alcohol consumption. NAFLD is a metabolic disorder that represents a disease spectrum, ranging from steatosis (isolated fatty liver) without specific liver injury to nonalcoholic steatohepatitis (NASH) in which there is inflammation leading to scarring, fibrosis, and possibly cirrhosis (Dowman et al., Ailment Pharmacol Ther., 33(5): 525-540 (2011)). Risk factors include male gender, age, obesity, insulin resistance, and metabolic syndrome (Bellentani et al., Dig. Dis., 28: 155-161 (2010)). In the United States, NAFLD is the most common form of chronic liver disease, and likely will be the leading cause of liver transplantation worldwide by 2020 (Musso et al., Ann. Med., 43: 617-649 (2011)). It has been shown that psoriasis patients have an increased incidence of nonalcoholic fatty liver disease over controls (Van der Voort et al., J. Amer. Acad. Dermatol., 70: 517-524 (2014); and Prussick et al., J. Clin. Aesthet. Dermatol., 8(3): 43-45 (2015)). Patients with nonalcoholic fatty liver disease and psoriasis have more severe skin disease and are at higher risk of severe liver fibrosis than patients without psoriasis (Miele et al., J Hepatol., 51: 778-786 (2009)).

As discussed above, some individuals with NAFLD can develop nonalcoholic steatohepatitis (NASH), an aggressive form of fatty liver disease, which is marked by liver inflammation and may progress to cirrhosis, hepatocellular carcinoma, or liver failure (Dowman et al., supra; and Prussick et al., supra). This damage is similar to the damage caused by heavy alcohol use. The risk of developing NASH is more than 33 percent in obese people but less than five percent in lean people (Prussick et al., supra). The only way to distinguish whether a patient has fatty liver disease or the more severe NASH is by liver biopsy. High-risk patients for NASH that may be candidates for liver biopsy are those with metabolic syndrome, obesity (BMI>30), and diabetes (Dowman et al., supra). In some embodiments, the methods described herein may be used to treat NAFLD, NASH, or noncirrhotic NASH with liver fibrosis (NC-NASH+LF) that occur in patients who also suffer from psoriasis. In other embodiments, the disclosed methods may be used to treat NAFLD, NASH, or NC-NASH+LF in patients that do not suffer from psoriasis. In addition, the disclosed methods may be used to treat liver inflammation generally, particularly liver inflammation associated with NAFLD, NASH, or other subsets of NAFLD.

A composition comprising an effective amount of an IL-17RA-binding monoclonal antibody or antigen-binding fragment thereof can be administered to a mammal using standard administration techniques, including oral, intravenous, intraperitoneal, subcutaneous, pulmonary, transdermal, intramuscular, intranasal, buccal, sublingual, or suppository administration. The composition preferably is suitable for parenteral administration. The term “parenteral,” as used herein, includes intravenous, intramuscular, subcutaneous, rectal, vaginal, and intraperitoneal administration. Ideally, the composition is administered to a mammal using peripheral systemic delivery by intravenous, intraperitoneal, or subcutaneous injection.

Dosing frequency will depend upon the pharmacokinetic parameters of the particular IL-17RA antigen binding protein in the formulation used. Typically, a clinician will administer the composition until a dosage is reached that achieves the desired effect. The composition therefore may be administered as a single dose, or as two or more doses (which may or may not contain the same amount of the monoclonal antibody) over time, or as a continuous infusion via an implantation device or catheter. The method of the present disclosure is not limited to a particular dosing frequency. In some embodiments, the composition may be administered once daily, but preferably is administered at least once a week for a therapeutic period. In other embodiments, the method may initially comprise once weekly administration of the composition followed by administration of the composition once every two weeks. For example, the composition may be administered once a week for three weeks, and then every two weeks for 12 weeks, for a total therapeutic period of 15 weeks. Alternatively, continuous administrations may be performed at the starting date of the administration every other week.

The therapeutic period for administration of the composition comprising the IL-17RA-binding monoclonal antibody is not particularly limited, however, the therapeutic period is desirably 10 weeks or more, 30 weeks or more, or 52 weeks (i.e., 1 year) or more. In one embodiment, the therapeutic period is about 10-20 weeks (e.g., 11, 12, 13, 14, 15, 16, 17, 18, or 19 weeks). In addition, the therapeutic period may include a rest period. Further refinement of the appropriate dosage regimen and therapeutic period may be made by those of ordinary skill in the art. For example, a dose of the composition described herein may be administered by subcutaneous injection at time “0” (i.e., the first administration), at one week post time “0” (i.e., the second administration), at two weeks post time “0” (i.e., the third administration), and then administered every two weeks following the third administration. Administration of the composition every two weeks may be performed for twelve weeks, for a total therapeutic period of 15 weeks.

Once administered to a mammal (e.g., a cross-reactive human), the biological activity and therapeutic efficacy of an IL-17 antagonist, such as the disclosed IL-17RA-binding antibody or antigen-binding fragment thereof, can be measured by any suitable method known in the art. For example, the biological activity can be assessed by determining the levels of one or more liver enzymes in the subject, as increased liver enzymes are typically associated with liver inflammation and fibrosis. Thus, the method described herein desirably results in decreased levels of liver enzymes. Levels of liver enzymes may be reduced by any suitable amount as compared to an initial measurement made prior to commencement of the disclosed method (i.e., baseline). For example, the level of one or more liver enzymes may be reduced by 20% or more, 30% or more, 40% or more, 50% or more, 60% or more, 70% or more, 80% or more, 90% or more, or 100% as compared to baseline. Any suitable liver enzyme, or combination of enzymes, may be measured to assess therapeutic efficacy of the described method. In this respect, a standard hepatic panel includes measurement of total bilirubin, alanine transaminase (ALT), aspartate transaminase (AST), AST/ALT ratio, alkaline phosphatase (ALP), gamma glutamyl transpeptidase (GGT), and albumin. In some embodiments, the method described herein results in a decrease in the levels of aspartate aminotransferase (AST) and/or alanine aminotransferase (ALT) in the subject. Other biological markers (biomarkers) of inflammation or liver function may be measured to assess therapeutic efficacy, including, for example, gamma-glutamyl transferase (GGT), lactate dehydrogenase (LDH), C-reactive protein (CRP), apolipoprotein, G2-macroglobulin, hyaluronic acid (HA), haptoglobin, procollagen type III amino terminal propeptide (PIIINP), tissue inhibitor of metalloproteinases-1 (TIMP-1), α-2 Macroglobulin (A2M), hemoglobin A1c (HbA1c), fasting insulin levels, and lipids. In one embodiment, the method described herein results in a decrease in the level of c-reactive protein (CRP) in the subject. The level of CRP may be reduced by any suitable amount as compared to an initial measurement made prior to commencement of the disclosed method (i.e., baseline). For example, the level of CRP may be reduced by 20% or more, 30% or more, 40% or more, 50% or more, 60% or more, 70% or more, 80% or more, 90% or more, or 100% as compared to baseline

The IL-17 antagonist, such as an IL-17RA-binding antibody or antigen binding fragment thereof, may be administered alone or in combination with other drugs or agents. For example, an IL-17 antagonist can be administered in combination with other agents for the treatment or prevention of NAFLD or NASH. In this respect, the IL-17 antagonist can be used in combination with at least one other anti-inflammatory agent including, for example, corticosteroids (e.g., prednisone and fluticasone), non-steroidal anti-inflammatory drugs (NSAIDs) (e.g., aspirin, ibuprofen, and naproxen), and other biologics (e.g., infliximab, adalimumab, etanercept, alefacept, ustekinumab, ixekizumab, secukinumab, and/or guselkumab). The additional agent or drug may be administered prior to, concurrent with, or subsequent to administration of the composition comprising the IL-17 antagonist.

The following examples further illustrate the invention but, of course, should not be construed as in any way limiting its scope.

### EXAMPLE 1

This example describes a pooled 48-week analysis of brodalumab on a marker of inflammation in psoriasis patients with potential indicators of early nonalcoholic fatty liver disease (NAFLD).

The objective of this study was to assess changes in the inflammatory marker C-reactive protein (CRP) in patients with psoriasis and early NAFLD indicators receiving brodalumab.

Data were pooled from two identically designed, randomized, double-blind psoriasis trials. Adults received subcutaneous administration of brodalumab (210 mg every 2 weeks) or ustekinumab (therapeutic human immunoglobulin (Ig) G1 kappa mAb that binds to the interleukins (IL)-12 and IL-2) 45 mg (body weight ≤100 kg) or 90 mg (>100 kg) at baseline, week 4, and then every 12 weeks for up to 48 weeks. At week 16, ustekinumab-treated patients with inadequate efficacy response could switch to brodalumab (210 mg every 2 weeks). CRP changes were analyzed in patients subgrouped by baseline fibrosis indicators (aspartate aminotransferase (AST)/alanine aminotransferase (ALT) ratio≥1.4, AST>40 U/L, fibrosis-4 (FIB4) score>1.3).

In the AST/ALT≥1.4 subgroup, CRP decreased significantly from baseline in the ustekinumab/brodalumab treatment group (16.2 mg/L (n=6)) versus the ustekinumab only treatment group (2.8 mg/L (n=56); P=0.01) at week 48. Additionally, the CRP decrease from baseline was numerically greater with ustekinumab/brodalumab versus ustekinumab in the AST>40 (3.3 (n=10) vs 0.1 mg/L (n=41)) and FIB4 score>1.3 (2.3 (n=18) vs 0.8 mg/L (n=85)) subgroups.

This long-term post hoc observation of decreased CRP levels in an early NAFLD population treated with brodalumab suggests that brodalumab may have activity in reducing liver inflammation.

### EXAMPLE 2

This example describes a phase 2a clinical study to evaluate the safety and efficacy of brodalumab in subjects with noncirrhotic nonalcoholic steatohepatitis with liver cirrhosis (NC-NASH+LF).

A multicenter randomized, double blind, placebo controlled, 24-week, phase 2a study will assess the anti-inflammatory and anti-fibrotic effects, and the safety and tolerability, of brodalumab compared to placebo in subjects with NC-NASH+LF. Subjects with NC-NASH+LF will be identified according to American Association for the Study of Liver Diseases (AASLD) criteria, VCTE-estimated F1-F3 fibrosis (2.88-4.67 kPa), magnetic resonance imaging (MRI)-proton density fat fraction (PDFF)-estimated liver fat >5%, body mass index (BMI)>25 kg/m², and elevated liver enzymes (ALT>30 and >19 in men and women, respectively).

After informed consent is obtained, blood will be drawn to determine if subjects meet the criteria for inclusion in the study. All labs during this study will be drawn in a fasting state and sent to the study-specified central laboratory for processing. Women of childbearing potential will undergo a serum or urine pregnancy test.

Subjects with elevated liver enzymes will be recruited as one of the outcomes being measured is improvement in liver enzymes. For evidence of liver inflammation and liver synthetic function, labs will include: complete blood count (CBC) with differential, basic metabolic profile (BMP), prothrombin time (PT)/INR, hepatic panel, gamma-glutamyl transferase (GGT), lactate dehydrogenase (LDH), C-reactive protein (CRP), apolipoprotein, G2-macroglobulin, hyaluronic acid (HA), haptoglobin, procollagen type III amino terminal propeptide (PIIINP), tissue inhibitor of metalloproteinase-1 (TIMP-1), and α 2 macroglobulin (A2M). A metabolic work-up also will be done, including hemoglobin A1c (HbA1c), fasting insulin levels, and a lipid profile. Insulin resistance will be calculated using the Homeostatic Model Assessment of Insulin Resistance (HOMA-IR). Blood also will be drawn to rule out other causes of liver disease (e.g., viral hepatitis, hereditary hemochromatosis, autoimmune liver disease, alpha-1-antitrypsin deficiency, and Wilson's disease when clinically appropriate). If the subject does not have a liver scan from within the 6-months prior to providing informed consent, blood will be drawn for alpha-fetoprotein (AFP) to rule out HCC.

The following composite scores will be calculated from the available blood work to establish a baseline degree of fibrosis: AST/ALT ratio, AST/platelet ratio index (APRI) score, BAAT score, BARD score, enhanced liver fibrosis (ELF) test, Fibrometer, Fibrosis-4 (FIB-4) score, Hepascore, and nonalcoholic fatty liver disease (NAFLD) fibrosis score (NFS).

Blood will be drawn for exploratory endpoints (Pro-C3 (an N-terminal type III collagen propeptide, a fibrosis marker), as well as CCR2 and CCRS (proinflammatory and profibrotic markers)).

To assess liver stiffness and the extent of hepatic steatosis, each subject will undergo VCTE with controlled attenuation parameter (CAP), MRI PDFF, and a Multiscan (where available).

Every patient will have a complete physical exam, structured interview (to collect data on alcohol consumption, prescription and over the counter medications, and herbal supplements), and review of his/her imaging of the liver and medical record.

A total of 60 subjects at approximately 10 clinical sites within the United States, who have been diagnosed with NAFLD, and who meet all inclusion criteria and none of the exclusion criteria will be enrolled in this clinical investigation. The estimated time of study duration, from the start of enrollment to completion of the EOS assessments for the final subject is 52 weeks. For each subject, the estimated time on study medication, from baseline to EOT Visit is 15 weeks. Subjects who meet the eligibility criteria and have given written informed consent will be randomized in a 1:1 allocation to one of two treatment groups. One group will receive brodalumab, and the other group will receive placebo. At each visit, adverse events (AEs) and serious adverse events (SAEs) will be recorded, and the medical record and concomitant medications will be reviewed.

Brodalumab will be provided in single-use pre-filled syringes containing 1.5 mL (210 mg) of brodalumab, formulated with 10 mM L-glutamate, 3% (w/v) L-proline, and 0.001% (w/v) polysorbate 20, at pH 4.8 each. The syringes have an attached 27G ½ inch needle and will be over-labeled with study-specific information. The proposed dosing regimen for the active arm of this phase 2 study is: brodalumab 210 mg SC once a week for three weeks, then once every two weeks for 12 weeks. The total duration of the proposed treatment is 15 weeks. Brodalumab will be compared to a placebo consisting of sterile saline for injection. The placebo will be administered in a dosing regimen matched to the brodalumab dosing regimen. The sterile saline will be supplied in 10 mL single-dose vials, and using the 27G ½ inch needles provided, 1.5 mL will be drawn up into 3 mL syringes using sterile technique.

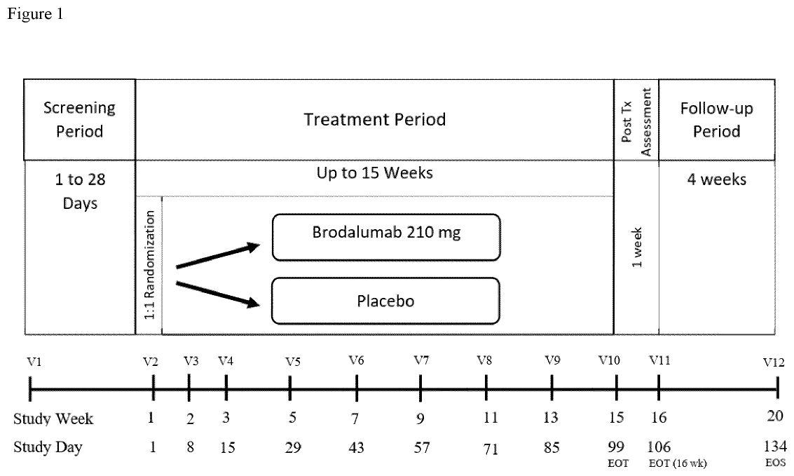

Labs will be repeated every four weeks throughout the treatment period. One week after the end of treatment (EOT (16 weeks)), subjects will undergo fasting blood work (CBC with differential, BMP, PT/INR, serum or urine pregnancy test (for women of childbearing potential), GGT, LDH, CRP, apolipoprotein, G2-macroglobulin, HA, haptoglobin, PIIINP, TIMP-1, A2M, HbA1c, fasting insulin levels, and lipid profile). Insulin resistance will again be calculated using HOMA-IR. Blood work for exploratory outcomes will also be drawn. A schematic of the clinical study is shown in FIG. 1.

The primary efficacy endpoint is improvement of the liver enzymes (AST and ALT) at EOT (16 weeks) compared to baseline. Exploratory endpoints include the following: (a) improvement in liver fat at 16 weeks following treatment with brodalumab, compared to baseline. Liver fat will be measured by MRI-PDFF, a non-invasive marker of liver fat, and by VCTE with CAP; (b) improvement in liver fibrosis at 16 weeks following treatment with brodalumab, compared to baseline. This will be measured by multiple non-invasive tools including Multiscan (where available), VCTE, and calculations of AST/ALT ratio, APRI score, BAAT score, BARD score, ELF test, FIB-4 score, Fibrometer, Hepascore, and NFS, (c) change in Pro-C3 (N-terminal type III collagen propeptide, a fibrosis marker) at 16 weeks following treatment with brodalumab, compared to baseline; (d) change in CCR2 and CCR5 (proinflammatory and profibrotic markers) at 16 weeks following treatment with brodalumab, compared to baseline. For all exploratory endpoints, mean change from baseline to EOT (16 weeks) will be summarized by treatment group. Treatment difference for mean change from baseline will be tested using ANCOVA model with effects for treatment and study center, and baseline value as a covariate.

Safety endpoints include (a) incidence of treatment-emergent adverse events (TEAEs) and serious adverse events (SAEs); (b) changes from baseline in clinical laboratory results, and (c) changes from baseline in vital signs. Safety evaluations will be based on the incidence, intensity and types of adverse events (AEs), and changes in vital signs and clinical laboratory results.

In general, statistical testing will be 2-sided at the p=0.05 level of significance. All continuous variables will be summarized using descriptive statistics; N, mean, standard deviation, median, maximum, and minimum. All categorical variables will be summarized using frequency counts and percentages. Baseline value is defined as last available value prior to the first dose of study drug. End of treatment value (EOT (16weeks) value) is defined as labs and assessments performed after the treatment period, at week 16.

SEQUENCES

SEQ ID NO: 1 (VH CDR1): RYGIS
SEQ ID NO: 2 (VH CDR2): WISTYSGNTNYAQKLQ
SEQ ID NO: 3 (VH CDR3): RQLYFDY
SEQ ID NO: 4 (VL CDR1): RASQSVSSNLA
SEQ ID NO: 5 (VL CDR2): DASTRAT
SEQ ID NO: 6 (VL CDR3): QQYDNWPLT
SEQ ID NO: 7 (VH):
QVQLVQSGAEVKKPGASVKVSCKASGYTFTRYGISWVRQAPGQGLEWMGWISTYSGN
TNYAQKLQGRVTMTTDTSTSTAYMELRSLRSDDTAVYYCARRQLYFDYWGQGTLVTV
SS
SEQ ID NO: 8 (VL):
EIVMTQSPATLSVSPGERATLSCRASQSVSSNLAWFQQKPGQAPRPLIYDASTRATGVPA
RFSGSGSGTDFTLTISSLQSEDFAVYYCQQYDNWPLTFGGGTKVEIK
SEQ ID NO: 9(IL-17RA):
MGAARSPPSAVPGPLLGLLLLLLGVLAPGGASLRLLDHRALVCSQPGLNCTVKNSTCLD
DSWIHPRNLTPSSPKDLQIQLHFAHTQQGDLFPVAHIEWTLQTDASILYLEGAELSVLQL
NTNERLCVRFEFLSKLRHHHRRWRFTFSHFVVDPDQEYEVTVHHLPKPIPDGDPNHQSK
NFLVPDCEHARMKVTTPCMSSGSLWDPNITVETLEAHQLRVSFTLWNESTHYQILLTSFP
HMENHSCFEHMHHIPAPRPEEFHQRSNVTLTLRNLKGCCRHQVQIQPFFSSCLNDCLRHS
ATVSCPEMPDTPEPIPDYMPLWVYWFITGISILLVGSVILLIVCMTWRLAGPGSEKYSDD
TKYTDGLPAADLIPPPLKPRKVWIIYSADHPLYVDVVLKFAQFLLTACGTEVALDLLEEQ
AISEAGVMTWVGRQKQEMVESNSKIIVLCSRGTRAKWQALLGRGAPVRLRCDHGKPV
GDLFTAAMNMILPDFKRPACFGTYVVCYFSEVSCDGDVPDLFGAAPRYPLMDRFEEVY
FRIQDLEMFQPGRMHRVGELSGDNYLRSPGGRQLRAALDRFRDWQVRCPDWFECENL
YSADDQDAPSLDEEVFEEPLLPPGTGIVKRAPLVREPGSQACLAIDPLVGEEGGAAVAKL
EPHLQPRGQPAPQPLHTLVLAAEEGALVAAVEPGPLADGAAVRLALAGEGEACPLLGSP
GAGRNSVLFLPVDPEDSPLGSSTPMASPDLLPEDVREHLEGLMLSLFEQSLSCQAQGGCS
RPAMVLTDPHTPYEEEQRQSVQSDQGYISRSSPQPPEGLTEMEEEEEEEQDPGKPALPLS
PEDLESLRSLQRQLLFRQLQKNSGWDTMGSESEGPSA
SEQ ID NO: 10 (Brodalumab Heavy Chain):
QVQLVQSGAEVKKPGASVKVSCKASGYTFTRYGISWVRQAPGQGLEWMGWISTYSGN
TNYAQKLQGRVTMTTDTSTSTAYMELRSLRSDDTAVYYCARRQLYFDYWGQGTLVTV
SSASTKGPSVFPLAPCSRSTSESTAALGCLVKDYFPEPVTVSWNSGALTSGVHTFPAVLQ
SSGLYSLSSVVTVPSSNFGTQTYTCNVDHKPSNTKVDKTVERKCCVECPPCPAPPVAGPS
VFLFPPKPKDTLMISRTPEVTCVVVDVSHEDPEVQFNWYVDGVEVHNAKTKPREEQFNS
TFRVVSVLTVVHQDWLNGKEYKCKVSNKGLPAPIEKTISKTKGQPREPQVYTLPPSREE
MTKNQVSLTCLVKGFYPSDIAVEWESNGQPENNYKTTPPMLDSDGSFFLYSKLTVDKSR
WQQGNVFSCSVMHEALHNHYTQKSLSLSPGK
SEQ ID NO: 11 (Brodalumab Light Chain):
EIVMTQSPATLSVSPGERATLSCRASQSVSSNLAWFQQKPGQAPRPLIYDASTRATGVPA
RFSGSGSGTDFTLTISSLQSEDFAVYYCQQYDNWPLTFGGGTKVEIKRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGEC

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating nonalcoholic fatty liver disease (NAFLD) in a subject, which comprises administering to the subject a composition comprising a therapeutically effective amount of a monoclonal antibody that specifically binds to interleukin 17 receptor A (IL-17RA), or an antigen-binding fragment thereof, and a pharmaceutically acceptable carrier, whereby the NAFLD is treated in the subject.

2. The method of claim 1, wherein the subject suffers from nonalcoholic steatohepatitis (NASH).

3. The method of claim 2, wherein the subject suffers from noncirrhotic nonalcoholic steatohepatitis with liver fibrosis (NC-NASH+LF).

4. A method of reducing liver inflammation in a subject in need thereof, which comprises administering to the subject a composition comprising a therapeutically effective amount of a monoclonal antibody that specifically binds to interleukin 17 receptor A (IL-17RA), or an antigen-binding fragment thereof, and a pharmaceutically acceptable carrier, whereby liver inflammation in the subject is reduced.

5. The method of claim 4, wherein the subject suffers from nonalcoholic fatty liver disease (NAFLD) or nonalcoholic steatohepatitis (NASH).

6. The method of any one of claims 1-5, wherein the monoclonal antibody comprises:
(a) a heavy chain variable region comprising a complementarity determining region 1 (CDR) amino acid sequence of SEQ ID NO: 1, a CDR2 amino acid sequence of SEQ ID NO: 2, and a CDR3 amino acid sequence of SEQ ID NO: 3; and
(b) a light chain variable region comprising a complementarity determining region 1 (CDR) amino acid sequence of SEQ ID NO: 4, a CDR2 amino acid sequence of SEQ ID NO: 5, and a CDR3 amino acid sequence of SEQ ID NO: 6.

7. The method of claim 6, wherein the monoclonal antibody comprises a heavy chain variable region amino acid sequence of SEQ ID NO: 7 and a light chain variable region amino acid sequence of SEQ ID NO: 8.

8. The method of any one of claims 1-7, wherein the subject suffers from psoriasis.

9. The method of any one of claims 1-8, wherein the composition comprises about 150 mg to about 250 mg of the monoclonal antibody.

10. The method of claim 9, wherein the composition comprises about 210 mg of the monoclonal antibody.

11. The method of any one of claims 1-10, wherein the composition comprises about 210 mg of brodalumab formulated with about 10 mM L-glutamate, about 3% (w/v) L-proline, and about 0.001% (w/v) polysorbate 20, and the pH of the composition is about 4.8.

12. The method of any one of claims 1-11, wherein the composition is administered at least once a week in a therapeutic period.

13. The method of claim 12, wherein the composition is administered once a week for three weeks, followed by once every two weeks for 12 weeks in a therapeutic period of 15 weeks.

14. The method of any one of claims 1-13, wherein the method results in a decrease in the levels of liver enzymes and/or a decrease in the level of c-reactive protein (CRP) in the subject.

15. The method of claim 14, wherein the method results in a decrease in the levels of aspartate aminotransferase (AST) and/or alanine aminotransferase (ALT) in the subject.

16. A monoclonal antibody that specifically binds to interleukin 17 receptor A (IL-17RA), or an antigen-binding fragment thereof, for use in treating nonalcoholic fatty liver disease (NAFLD) in a subject, wherein the antibody or antigen-binding fragment thereof is comprised in a composition comprising a pharmaceutically acceptable carrier, and wherein the antibody or antigen-binding fragment thereof is preferably administered to the subject in a therapeutically effective amount.

17. The monoclonal antibody or antigen-binding fragment thereof for use according to claim 16, wherein the subject suffers from nonalcoholic steatohepatitis (NASH).

18. The monoclonal antibody or antigen-binding fragment thereof for use according to claim 17, wherein the subject suffers from noncirrhotic nonalcoholic steatohepatitis with liver fibrosis (NC-NASH+LF).

19. A monoclonal antibody that specifically binds to interleukin 17 receptor A (IL-17RA), or an antigen-binding fragment thereof, for use in reducing liver inflammation in a subject, wherein the antibody or antigen-binding fragment thereof is comprised in a composition comprising a pharmaceutically acceptable carrier, and wherein the antibody or antigen-binding fragment thereof is preferably administered to the subject in a therapeutically effective amount.

20. The monoclonal antibody or antigen-binding fragment thereof for use according to claim 19, wherein the subject suffers from nonalcoholic fatty liver disease (NAFLD) or nonalcoholic steatohepatitis (NASH).

21. The monoclonal antibody or antigen-binding fragment thereof for use according to any one of claims 16 to 20, wherein the monoclonal antibody comprises:
(a) a heavy chain variable region comprising a complementarity determining region 1 (CDR) amino acid sequence of SEQ ID NO: 1, a CDR2 amino acid sequence of SEQ ID NO: 2, and a CDR3 amino acid sequence of SEQ ID NO: 3; and
(b) a light chain variable region comprising a complementarity determining region 1 (CDR) amino acid sequence of SEQ ID NO: 4, a CDR2 amino acid sequence of SEQ ID NO: 5, and a CDR3 amino acid sequence of SEQ ID NO: 6.

22. The monoclonal antibody or antigen-binding fragment thereof for use according to claim 21, wherein the monoclonal antibody comprises a heavy chain variable region amino acid sequence of SEQ ID NO: 7 and a light chain variable region amino acid sequence of SEQ ID NO: 8.

23. The monoclonal antibody or antigen-binding fragment thereof for use according to any one of claims 16 to 22, wherein the subject suffers from psoriasis.

24. The monoclonal antibody or antigen-binding fragment thereof for use according to any one of claims 16 to 23, wherein the composition comprises about 150 mg to about 250 mg of the monoclonal antibody.

25. The monoclonal antibody or antigen-binding fragment thereof for use according to claim 24, wherein the composition comprises about 210 mg of the monoclonal antibody.

26. The monoclonal antibody or antigen-binding fragment thereof for use according to any one of claims 16 to 25, wherein the composition comprises about 210 mg of brodalumab formulated with about 10 mM L-glutamate, about 3% (w/v) L-proline, and about 0.001% (w/v) polysorbate 20, and the pH of the composition is about 4.8.

27. The monoclonal antibody or antigen-binding fragment thereof for use according to any one of claims 16 to 26, wherein the composition is administered at least once a week in a therapeutic period.

28. The monoclonal antibody or antigen-binding fragment thereof for use according to claim 27, wherein the composition is administered once a week for three weeks, followed by once every two weeks for 12 weeks in a therapeutic period of 15 weeks.

29. The monoclonal antibody or antigen-binding fragment thereof for use according to any one of claims 16 to 28, wherein the use results in a decrease in the levels of liver enzymes and/or a decrease in the level of c-reactive protein (CRP) in the subject.

30. The monoclonal antibody or antigen-binding fragment thereof for use according to claim 29, wherein the use results in a decrease in the levels of aspartate aminotransferase (AST) and/or alanine aminotransferase (ALT) in the subject.


SEQUENCE LISTING (11 sequences)

SEQ ID NO: 1 - protein, 5 aa, Artificial Sequence
  VH CDR1
RYGIS

SEQ ID NO: 2 - protein, 16 aa, Artificial Sequence
  VH CDR2
WISTYSGNTNYAQKLQ

SEQ ID NO: 3 - protein, 7 aa, Artificial Sequence
  VH CDR3
RQLYFDY

SEQ ID NO: 4 - protein, 11 aa, Artificial Sequence
  VL CDR1
RASQSVSSNLA

SEQ ID NO: 5 - protein, 7 aa, Artificial Sequence
  VL CDR2
DASTRAT

SEQ ID NO: 6 - protein, 9 aa, Artificial Sequence
  VL CDR3
QQYDNWPLT

SEQ ID NO: 7 - protein, 116 aa, Artificial Sequence
  VH
QVQLVQSGAEVKKPGASVKVSCKASGYTFTRYGISWVRQAPGQGLEWMGWISTYSGNTNYAQKLQGRVTMTTDTSTSTAYMELRSLRSDDTAVYYCARRQLYFDYWGQGTLVTVSS

SEQ ID NO: 8 - protein, 107 aa, Artificial Sequence
  VL
EIVMTQSPATLSVSPGERATLSCRASQSVSSNLAWFQQKPGQAPRPLIYDASTRATGVPARFSGSGSGTDFTLTISSLQSEDFAVYYCQQYDNWPLTFGGGTKVEIK

SEQ ID NO: 9 - protein, 866 aa, Artificial Sequence
  IL-17RA
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWVYWFITGISILLVGSVILLIVCMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 10 - protein, 442 aa, Artificial Sequence
  Brodalumab Heavy Chain
QVQLVQSGAEVKKPGASVKVSCKASGYTFTRYGISWVRQAPGQGLEWMGWISTYSGNTNYAQKLQGRVTMTTDTSTSTAYMELRSLRSDDTAVYYCARRQLYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 11 - protein, 214 aa, Artificial Sequence
  Brodalumab Light Chain
EIVMTQSPATLSVSPGERATLSCRASQSVSSNLAWFQQKPGQAPRPLIYDASTRATGVPARFSGSGSGTDFTLTISSLQSEDFAVYYCQQYDNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC